(19) United States
(12) Patent Application Publication
(10) Pub. No.: US 2026/0358227 A1
(43) Pub. Date: Nov. 12, 2026

(54) 3D IN VITRO PERMEATION MODEL

(71) Applicant: LTS Lohmann Therapie-Systeme AG, Andernach (DE)

(72) Inventors: Julia LODDER-GADACZEK, Selters (DE); Michelle BAMBERG, Schweich (DE); Tanja UHRMACHER, Waldesch (DE); Julia KISTNER, Andernach (DE)

(21) Appl. No.: 19/134,486

(22) PCT Filed: Dec. 1, 2023
(86) PCT No.: PCT/EP2023/083975

§ 371 Date: May 30, 2025

(30) Foreign Application Priority Data
Dec. 2, 2022 (DE) 10 2022 132 085.5

(51) Int. Cl. G01N 15/08; G01N 33/483

(52) U.S. Cl. CPC G01N 15/0806; G01N 33/4833; G01N 2015/086

(57) ABSTRACT

The present invention relates to a permeation cell having a hollow body with at least two openings, characterized in that each of the at least two openings is closed by at least one semi-permeable membrane, to a device for measuring permeation, to a method for measuring permeation, and to the use of such a permeation cell for determining permeation.

The present invention relates to a permeation cell, a device for measuring a permeation, a method for measuring a permeation, and the use of such a permeation cell for determining a permeation.

Permeation cells are generally closed containers made of a dimensionally stable material with at least one opening that is closed by a permeable, in particular a semi-permeable, membrane. The permeation cell is filled with a medium which itself or at least one substance contained or dissolved therein can diffuse through the permeable or semi-permeable membrane. The medium with which the permeation cell is filled is not further limited and comprises solutions, dispersions, suspensions, or (condensed) gases of various compositions. Permeation cells are often used to determine the permeability of the membrane to be tested. During the permeation measurement, the at least one permeable membrane of the permeation cell is preferably in contact with the medium inside the permeation cell on the one hand and with a medium outside the permeation cell on the other. The permeation is determined, e.g., by determining the change in concentration of the permeate. The type of membrane to be tested is not limited and therefore is used in a wide range of applications. In the medical field in particular, the membrane can comprise, for example, animal or human skins.

In pharmacology, oral administration of drug-containing thin films is widely used as an alternative drug formulation, so it may be of interest to determine the permeability coefficients of drugs released from oral thin films.

Oral thin films (OTF) are thin, films mostly containing an active pharmaceutical ingredient, which are placed directly in the oral cavity or applied to the oral mucosa, where they dissolve. In particular, they are thin polymer-based films containing active ingredients which, when applied to a mucous membrane, especially the oral mucosa, release the active ingredient directly into it. These oral thin films are generally not tacky on the outside. The excellent blood circulation of the oral mucosa ensures that the active ingredient is quickly absorbed into the bloodstream. This dosage system has the advantage that the active ingredient is largely absorbed through the mucous membrane, thus avoiding the “first-pass metabolism” that occurs with the conventional dosage form of an active ingredient in tablet form, which is usually taken with liquid, which can be disadvantageous. The active ingredient can be dissolved, emulsified, or dispersed in the film.

Until now, standard in vitro models, e.g. the Franz cell, have not been able to correctly reproduce the permeation of active substances into or through the mucosa under conditions prevailing in reality. It should be noted that the OTF in the oral cavity is subject to muscle and tongue movements, which lead to an increased actual absorption surface. As a result, the dissolution time of the OTFs determined in conventional models is slower and the uptake of active substance is lower than observed under real conditions. This deviation is mainly due to the fact that the available surface area and the amount of medium of the standard in vitro model are too small. Furthermore, standard in vitro models have some preparative disadvantages such as the cumbersome attachment of the semi-permeable membrane, the insertion of the test specimen, e.g. an OTF, through the mostly narrow opening of the standard in vitro models and the frequent changing of the medium outside the permeation cell due to the short replacement times.

The present invention was therefore based on the problem of eliminating the disadvantages of the prior art depicted above. In particular, the present invention was based on the problem of providing a permeation cell which has a larger permeation surface and is additionally capable of accommodating a higher volume of medium than previous standard in vitro models. Further, the conditions particularly prevailing in a patient's mouth, i.e. the fact that the OTF in the oral cavity is subject to muscle and tongue movements, which leads to an increased actual absorption surface, should be mimicked as realistically as possible. In addition, the handling should be improved in that the permeation cell can be filled more quickly due to the easily accessible openings, and the semi-permeable membranes can be fastened to the permeation cell more easily.

These problems were solved by a permeation cell according to claim 1, i.e. by a permeation cell comprising a hollow body with at least two openings, which is characterized in that the at least two openings are each closed by at least one semi-permeable membrane.

Such a permeation cell has the advantage that the diffusion surface is considerably increased by a 3-dimensional arrangement of the membrane and that a significantly larger amount of medium can be used due to the available volume, so that an in vitro permeation measurement that better corresponds to the actual conditions is possible. Furthermore, handling has been improved to the effect that the permeation cell can be filled more quickly due to the easily accessible openings.

The terminology used in the description of the present disclosure is intended only to describe certain embodiments and should not be construed as limiting the subject matter. As used in the present description and claims, the singular forms “a”, “an” are to be understood to include the plural forms as well, unless the context clearly dictates otherwise. This also applies vice versa, i.e., the plural forms also include the singular forms. It is also understood that the term “and/or” as used herein refers to and includes all possible combinations of one or more of the associated listed elements. Moreover, it is to be understood that the terms “include”, “including”, “comprise”, and/or “comprising”, when used in the present description and claims, specify the presence of the specified features, steps, elements, and/or components, but do not exclude the presence or addition of one or more other features, steps, elements, components, and/or groups thereof.

In the present description and claims, the terms “include”, “comprise”, and/or “comprising” may also mean “consisting of”, i.e., the presence or addition of one or more other features, steps, elements, components, and/or groups is excluded.

The present invention relates to a permeation cell comprising a hollow body with at least two openings, wherein the at least two openings are each closed by at least one semi-permeable membrane.

In particular, a hollow body is understood to refer to a body whose interior is hollow, resulting in a volume that can be filled. The shape of the hollow body can be designed in different ways and is not subject to any restrictions. In particular, there does not have to result a continuous lateral surface, so that a hollow body is also not limited in the number of existing openings and consequently the number of openings can be adapted to the respective technical requirements.

The at least two openings of the hollow body are preferably designed so that they can be closed, in particular with a semi-permeable membrane, which is preferably held in position by corresponding holding devices. Suitable holding devices are, for example, elastic bands or rings, but clamps, adhesive tapes or other adhesive devices are suitable as well. It is also possible to close several openings by one and the same semi-permeable membrane. In doing so, the surfaces of the openings are preferably completely covered so that a continuous surface of hollow body and closed opening is created. On the one hand, the corresponding holding devices can ensure that the semi-permeable membrane is held securely in place, and on the other hand they also allow the hollow body to be reversibly opened and closed. The holding device can be designed in different ways, wherein it should be ensured that the semi-permeable membrane to be attached is not damaged during attachment and removal and that continuous contact of the semi-permeable membrane with the edges of the opening is guaranteed. Furthermore, the holding devices can be mounted in such a way that a semi-permeable membrane covers several openings.

A semi-permeable membrane generally refers to a partly permeable membrane that allows molecules from an adjacent medium to selectively diffuse through it (permeate), while others are retained (retentate). Films of synthetic polymers, cell membranes, parchment, or animal or human skins are used as semi-permeable membranes.

The passage of substances takes place according to the general principle of permeation, wherein the molecules sorb at the surface of the semi-permeable membrane, diffuse to the other side and desorb there again. Without external influences, the permeate always moves in the direction of the lower concentration, more precisely along the gradient of the chemical potential of the permeate.

The permeation cell according to the invention is further preferably characterized in that the at least one semi-permeable membrane comprises a mucous membrane.

A mucous membrane (tunica mucosa or mucosa for short) refers to a protective layer that lines the inside of hollow organs. Mucous membranes are located in the oral and nasal cavities, among other places. In contrast to the external skin, the mucous membrane has neither a horny layer nor hair. The function of the mucous membrane is to prevent the penetration of foreign substances and the drying out of body tissue; additionally, most mucous membranes also secrete mucous substances. When using a mucous membrane as the closing membrane of the permeation cell according to the invention, it must be ensured in one embodiment that the epithelium of the mucous membrane faces the interior of the permeation cell according to the invention.

The mucosa preferably comprises a human or animal mucosa, preferably a buccal mucosa or esophagus.

Membranes of different origin and thickness can be used for in vitro permeation measurements, wherein both animal and human mucosa are used. Overall, the mucosa from the Göttingen minipig has established itself as a good alternative to human mucosa due to comparable results and minor deviations in skin quality. The layer thickness of the skin resulting from different preparations varies between 0.5 and 1.2 mm.

The mucous membrane preferably comprises oral mucous membrane, in particular a buccal mucous membrane, particularly preferably the esophagus of the domestic pig or Göttingen minipig.

The permeation cell according to the invention is further preferably characterized in that the hollow body has a volume of from 1 to 5 cm³, preferably from 1 to 4 cm³, particularly preferably from about 1.5 to about 3 cm³.

The volume of the hollow body preferably corresponds to the hollow volume of the hollow body, wherein the volume is limited by the surface of the hollow body when the openings are closed.

The permeation cell according to the invention is further preferably characterized in that the total area of the semi-permeable membranes is from 2 to 12 cm², preferably from 2.5 to 10 cm², particularly preferably from about 3 to about 9 cm².

The total area of the semi-permeable membranes is the sum of the areas of the individual semi-permeable membranes. Here, the area of the individual membranes considered preferably corresponds to the area of the closed opening, so that the areas that are only used for fastening or protrude do not have to be considered.

The individual semi-permeable membrane is further preferably characterized in that it has a surface area of from 1 to 6 cm², preferably from 1 to 5 cm², particularly preferably from about 1.5 to about 4.5 cm³.

The permeation cell according to the invention is further preferably characterized in that the hollow body comprises a material selected from the group consisting of polymers, preferably polystyrene.

In principle, the material used is not subject to any restrictions as long as it does not affect the permeation process, is chemically inert, and sufficiently dimensionally stable.

The permeation cell according to the invention is further preferably characterized in that the hollow body has the shape of a hollow cylinder, a hollow parallelepiped, a hollow cube, a hollow pyramid, a hollow prism, a hollow cone, and/or a hollow sphere, or a shape derived from these.

The shape of a hollow cylinder corresponds in particular to that of a straight circular cylinder.

The other hollow bodies mentioned also essentially correspond to the general definitions with regard to their shape and determinants. Other shapes that can be derived from this comprise hollow bodies that can be derived from the aforementioned hollow bodies by simple adaptations, e.g. an oblique hollow cylinder. Furthermore, combinations of the listed bodies are also conceivable, such as, e.g., a hollow pyramid whose base is flush with the surface of a hollow cube.

The invention further relates to a device for measuring a permeation of molecules through at least one semi-permeable membrane, comprising the permeation cell according to the invention as a donor compartment, wherein the permeation cell contains a first medium comprising molecules, and an acceptor compartment containing a second medium, wherein the acceptor compartment is designed such that the second medium completely covers the semi-permeable membranes of the permeation cell.

In the context of the device according to the invention for measuring the permeation of molecules, the term molecules comprises all chemical compounds, ions or atoms that can diffuse through the at least one semi-permeable.

In particular, the donor compartment is inserted into the device in such a way that it is completely covered by the acceptor compartment. This applies in particular to the semi-permeable membranes. If necessary, the donor compartment can be secured or designed in different ways to ensure that it is completely covered even throughout the measurement, e.g. by securing it to a suitable position on the device.

The device according to the invention for measuring a permeation of molecules through at least one semi-permeable membrane is further preferably characterized in that the molecules comprise at least one pharmaceutically active ingredient.

The at least one pharmaceutically active ingredient is in principle not subject to any limitation, but is preferably selected from all pharmaceutically active ingredients that are suitable for oral and/or transmucosal administration.

According to the present invention, the pharmaceutically active ingredient also subsumes, for example, all pharmaceutically acceptable salts and solvates of the respective pharmaceutically active ingredient.

Preferred are active ingredients selected from the group comprising the active ingredient classes of analgesics, hormones, hypnotics, sedatives, antiepileptics, amphetamines, psychoneurotropic agents, neuromuscular-blocking agents, antispasmodics, antihistamines, antiallergics, cardiotonic agents, antiarrhythmics, diuretics, antihypertensives, vasopressors, antidepressants, antitussives, expectorants, thyroid hormones, sex hormones, antidiabetics, antitumor agents, antibiotics, chemotherapeutics and narcotics, wherein this group is not exhaustive.

Particularly preferably, the at least one pharmaceutically active ingredient is ketamine and/or a pharmaceutically acceptable salt or solvate thereof, preferably ketamine·HCl.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

The device according to the invention for measuring a permeation of molecules through at least one semi-permeable membrane is further preferably characterized in that the first medium comprising the molecules comprises a solution, dispersion, and/or suspension comprising at least one pharmaceutically active ingredient.

The first medium therefore comprises dissolved and/or undissolved molecules, in particular those that are capable of diffusing through the semi-permeable membrane.

The solvent and/or the liquid phase of the first medium preferably comprises or consists of water or a solution suitable to simulate the conditions in the oral cavity, wherein the use of water is preferred. Examples for solutions that are suitable for simulating the conditions in the oral cavity are physiological phosphate buffer, physiological phosphate buffer with thiocyanate, isotonic saline solution, human or animal saliva, or artificial human saliva (glandosanes or buffers).

The first medium is further preferably characterized in that said medium comprises the sample to be tested including the application of the OTF and an artificial saliva substitute solution, wherein said artificial saliva substitute solution preferably comprises potassium thiocyanate (KSCN), potassium chloride (KCl), sodium hydrogen phosphate hydrate (Na₂HPO₄*H₂O), mucins (for example mucin from porcine stomach type II from Sigma Aldrich), and demineralized water.

Furthermore, the first medium is further preferably characterized in that it is adjusted to a pH of 6 to 8 by the addition of an acid and/or a base, wherein the pH of 7 to 7.5, in each case at 37 °C, is particularly preferred.

The first medium may further comprise suitable excipients. In particular, common excipients such as permeation accelerators should be mentioned here.

The device according to the invention for measuring a permeation of molecules through at least one semi-permeable membrane is further preferably characterized in that the second medium comprises at least one solvent, a buffer, and/or a buffer with detergents, azides, or the like.

The second medium preferably comprises or consists of water or a mixture of water and one or more organic solvents, wherein the use of water is preferred. For example, acetonitrile would be a suitable organic solvent.

The second medium is further preferably characterized in that it comprises a pH-regulating buffer. The buffer is preferably selected from the group consisting of a solution with a physiological salt concentration, wherein a phosphate buffer is particularly preferred; the phosphate buffer is particularly preferably prepared from potassium phosphate and/or sodium dihydrogen phosphate.

The second medium may further comprise suitable excipients to improve the solubility of the active ingredient in the second medium. In particular, excipients such as cyclodextrins or surfactants should be mentioned here.

The device according to the invention for measuring a permeation of molecules through at least one semi-permeable membrane is further preferably characterized in that the donor compartment is designed such that it has a higher density in its entirety than the medium in the acceptor compartment.

The donor compartment is preferably characterized in that it has a higher density than the second medium in order to ensure, as described above, that it is completely covered by the second medium. This can be achieved, for example, by the first medium having a higher density than the second medium. Alternatively, the hollow body of the permeation cell or an additional material introduced into the permeation cell can result in a sufficiently high density or a sufficiently high weight of the donor compartment, so that the buoyancy force of the donor compartment is compensated or overcompensated.

Preferably, the hollow body comprises a material, such as a metal, which, when filled with the first medium, gives the hollow body as a whole a density so high that, when the permeation cell is placed in the acceptor compartment, the semi-permeable membranes are completely covered by the second medium.

Alternatively, small, for example spherical, particles of a high-density material can also be introduced into the permeation cell, i.e. into the volume of the hollow body, in order to give the permeation cell as a whole such a high density that the semi-permeable membranes are completely covered by the second medium when the permeation cell is placed in the acceptor compartment.

Small particles that may be considered here include, for example, metal beads with a diameter of around 1 to 8 mm, preferably around 2 to 6 mm. The metal beads are preferably made of stainless steel or lead.

The invention further relates to a method for measuring a permeation of molecules through at least one semi-permeable membrane, comprising the steps of:
providing the permeation cell according to the invention as a donor compartment, wherein the permeation cell contains a first medium comprising molecules,
providing an acceptor compartment comprising a second medium,
placing the permeation cell in the acceptor compartment so that the semi-permeable membranes of the donor compartment are completely covered with the second medium.

Providing the permeation cell according to the invention as a donor compartment preferably comprises removing one of the semi-permeable membranes at least partially and filling the permeation cell with the first medium. Preferably, the permeation cell according to the invention is filled to 70%, particularly preferably to 80%, relative to the volume of the hollow body of the permeation cell. The opening of the permeation cell is then preferably closed again with a semi-permeable membrane.

The incubation of the filled permeation cell according to the invention is preferably characterized in that the permeation cell is placed on a table shaker placed in the incubator at a temperature range from 25 to 38 °C, particularly preferably from 30 to 37 °C, and is rotated at 20 to 300 revolutions per minute (rpm), particularly preferably from 100 to 220 rpm, for 60 s to 30 min, particularly preferably for 3 to 10 min.

This procedure offers the advantage that the permeation cell according to the invention does not have to be completely filled, and thus an efficient homogenization of the first medium is made possible by the free volume, so that the soluble part of the OTF to be tested can be dissolved in the first medium in a short time.

Providing an acceptor compartment includes in particular the filling of a suitable container, e.g. a beaker, with the second medium.

The acceptor compartment is also preferably characterized in that it can be set to a temperature of 36 to 38, wherein the temperature of 37 °C is particularly preferred, and/or has a stirring unit.

Placing the permeation cell in the acceptor compartment preferably comprises placing the permeation cell in the acceptor compartment in such a way that, in particular, the semi-permeable membranes are completely covered by the acceptor compartment. If necessary, the donor compartment can be secured or designed in different ways to ensure that it is completely covered even throughout the measurement.

The method according to the invention for measuring a permeation of molecules through at least one semi-permeable membrane further preferably comprises the step of taking a sample from the acceptor compartment to determine a concentration of the molecules that have diffused through the semi-permeable membranes of the donor compartment.

The sample is taken from the acceptor compartment using a suitable device, such as a pipette. The amount of sample removed can be balanced by adding a medium, in particular according to the composition of the second medium. If the quantity of the sample taken is not balanced, this can also be compensated for by adjusting the calculation basis for the next sample taken.

The amount of permeated molecules in the sample taken can be determined using suitable measuring methods. Such measurement methods comprise, for example, HPLC methods, but also other quantitative analysis methods, for example based on mass spectrometry, NMR, IR, photometry, and/or titration.

The method according to the invention for measuring a permeation of molecules through at least one semi-permeable membrane is further preferably characterized in that the sampling takes place over a period of 0.5 to 360 min, wherein samples preferably are taken at regular intervals.

The method according to the invention for measuring a permeation of molecules is further preferably characterized in that samples are taken from the acceptor compartment at intervals of 1 to 60 min, for example at intervals of 3 min, 5 min, 10 min, or 15 min, starting with the placement of the permeation cell in the acceptor compartment.

The invention also relates to the use of the permeation cell according to the invention or the device according to the invention for determining a permeation of molecules through at least one semi-permeable membrane.

The preferred embodiments listed above for the permeation cell according to the invention also apply to the device according to the invention for measuring a permeation, the method for measuring a permeation and the use of such permeation cells for determining a permeation.

### DESCRIPTION OF THE FIGURES

FIG. 1: Schematic representation of a possible permeation cell according to the invention, wherein a straight hollow cylinder was used as the hollow body. The two openings are each closed with a mucosa as a semi-permeable membrane, wherein rubber bands were used as a holding device for the mucosa.

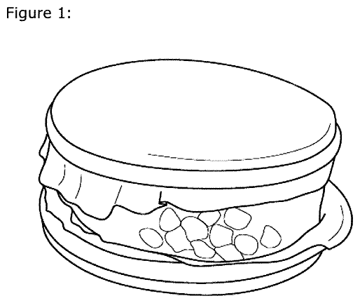

FIG. 2: Schematic representation of a conventional 2D mucosa model.

FIG. 3: Schematic representation of the device for measuring permeation through two mucosae. The donor compartment comprises a preferred embodiment of the permeation cell, which is completely covered by the acceptor compartment.

### LIST OF REFERENCE NUMBERS

1 Cell culture plate
2 Donor
3 Acceptor
4 Insert
5 Dissolved active ingredient
6 Mucosa
7 Prepared insert

### EXAMPLES

The invention is explained below in more detail with reference to non-limiting examples.

For optimization and the associated increase of the surface area of the 2D standard in vitro model for measuring the permeation of OTFs containing ketamine HCl, various models such as the 2D overlay model and the permeation cell according to the invention were developed and studied.

To determine the concentration, the samples were filtered and then analyzed using an HPLC system (Prominence-i LC-2030C 3D Plus, manufactured by Shimadzu®) under the parameters listed in Table 1.

TABLE 1
Parameters for the HPLC system

| Parameter: | Method: | |
|---|---|---|
| Mobile phase | phosphate buffer: methanol: | |
| | triethylamine, pH = 4.0 | |
| Stationary phase | modified silica gel (C18) | |
| Temperature column oven | 40 | °C |
| Temperature autosampler | 15 | °C |
| Flow rate | 0.7 | mL/min |
| Injection volume | 15 | μl |
| Detector | DAD: 220 nm | |
| Pressure | 140 | bar |
| Retention time | 4.5 | min |

### Example 1

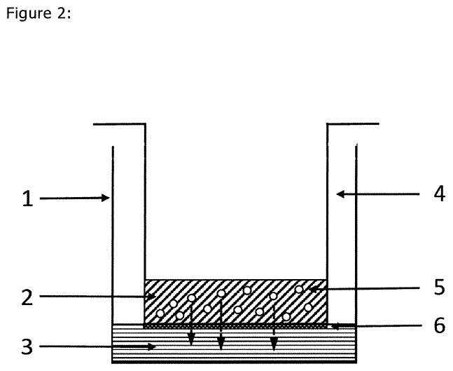

As a first example, the permeability of OTFs containing ketamine HCl was measured using the 2D standard in vitro model (analogous to FIG. 2).

For the 2D standard in vitro model, 12-well inserts were used in addition to 6-well inserts, which were covered with a punched piece of mucosa (4.524 cm²) and secured with a rubber band.

In order to determine how much ketamine HCl permeates through the mucosa, in vitro permeation tests were performed on mucosa. For this purpose, 1.7 mL of phosphate buffer (pH 7.4), which acted as an acceptor solution, was added to each well of a 6-well cell culture plate. Six cell culture plates were filled with the acceptor solution for the specific replacement times of 3, 6, 9, 12, 15, 18, 21, 24, 27 and 30 minutes and preheated at 37 °C in an incubator. Subsequently, five prepared mucosa 6-well inserts were each filled with 200 μL of diluted saliva substitute solution (donor solution) using a pipette and an OTF containing ketamine HCl was applied into each insert by means of a pair of tweezers. Then 300 UL of diluted saliva substitute solution was pipetted onto each OTF. To prepare the blank value, 500 μL of diluted saliva substitute solution was added to a prepared insert.

In order to start permeation and after filling the inserts in a storage plate, the first 6-well cell culture plate with the acceptor was removed from the incubator for the first 3-minute replacement time and one insert was transferred to each well by means of a pair of tweezers. The cell culture plate loaded with the 2D standard in vitro model was then incubated at 37 °C and 350 rpm in a thermoshaker for 3 min. In order to replace the acceptor after 3 minutes, the plate was removed from the thermoshaker and each insert was carefully transferred to a fresh well on a new, preheated 6-well plate using a pair of tweezers, and this procedure was continued for the subsequent replacements. The total duration of the incubation was 30 minutes, with a change of acceptor every 3 minutes. The collected samples were then analyzed using an HPLC system.

TABLE 2
Measured values from Example 1.

| Flux at | | | | Normalized |
|---|---|---|---|---|
| replacement | Average flux | Steady- | Permeability | permeated |
| time 3 minutes | (Jₐ) | state flux | coefficient | amount |
| [μg/cm² * min] | [μg/cm² * min] | (Jₛₛ) | (K_{P}) * | [μg/cm²] |
| 6.266 | 29.320 | 34.420 | 6.125 | 892 |

### Example 2

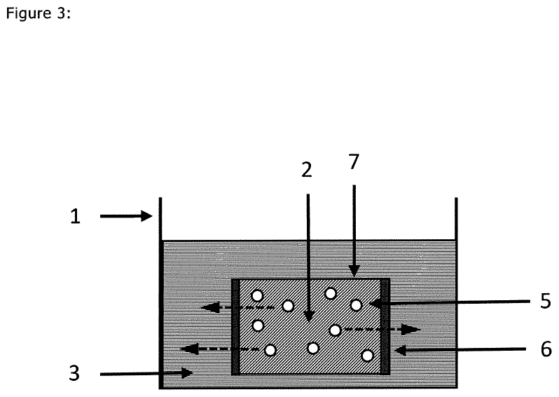

As a second example, the permeability of an OTF containing ketamine HCl was measured using the 2D overlay model (analogous to FIG. 3).

For the 2D overlay model, 12-well inserts were used in addition to 6-well inserts, which were covered with a punched piece of mucosa (4.524 cm²) and secured with a rubber band. In addition, another rubber band was pulled around the holders of the 6-well insert in the cell culture plate. This rubber band acted as a holder for the 12-well insert during permeation. In the prepared 12-well inserts, a punched piece of mucosa (1.131 cm²) was also pulled in reverse orientation so that the epithelium of the mucosa faced the outside of the insert and thus epithelium to epithelium with the 6-well insert.

After inserting the punched pieces of mucosa over a 6-well and a 12-well insert, six 6-well cell culture plates were filled with acceptor (phosphate buffer, pH 7.4) for the specific replacement times of 3, 6, 9, 12, 15, 18, 21, 24, 27 and 30 minutes and preheated in the incubator. The five 6-well inserts were loaded with diluted saliva substitute solution and an OTF containing ketamine HCl each. The blank value was again filled with 500 μL of diluted saliva substitute solution. In order to study the drug permeation in both directions, 1 mL of acceptor solution was also added to each of six wells of the 12-well inserts; the 12-well inserts were then attached to the rubber holder of the filled 6-well insert for permeation and transferred to a preheated 6-well cell culture plate using a pair of tweezers.

In order to start permeation and after filling the inserts in a storage plate, the first 6-well cell culture plate with the acceptor was removed from the incubator for the first 3-minute replacement time and one insert was transferred to each well by means of a pair of tweezers. The cell culture plate loaded with the inserts was then incubated at 37 °C and 350 rpm in a thermoshaker for 3 min. In order to replace the acceptor after 3 minutes, the plate was removed from the thermoshaker and each insert was carefully transferred to a fresh well on a new, preheated 6-well plate using a pair of tweezers, and this procedure was continued for the subsequent replacements. The total duration of the incubation was 30 minutes, with the acceptor being replaced every 3 minutes and, in addition to the complete removal of the acceptor from the cell culture plate, a partial removal of 0.5 mL from the acceptor solution of the respective 12-well insert was carried out by means of a pipette into a centrifuge tube at each replacement time. After partial sampling, 0.5 mL of fresh acceptor solution was pipetted into each insert. The collected samples were then analyzed using an HPLC system.

TABLE 3
Measured values from Example 2.

| Flux at | | | | Normalized |
|---|---|---|---|---|
| replacement | Average flux | Steady- | Permeability | permeated |
| time 3 minutes | (Jₐ) | state flux | coefficient | amount |
| [μg/cm² * min] | [μg/cm² * min] | (Jₛₛ) | (K_{P}) * | [μg/cm²] |
| 16.840 | 37.088 | 43.457 | 7.733 | 1136 |

### Example 3

As an example according to the invention, the permeability of an OTF containing ketamine HCl was measured using the device according to the invention (analogous to FIG. 4).

For the in vitro measurement of the permeation, the permeation cell according to the invention was used, wherein a straight hollow cylinder was used as a hollow body and the two openings were each closed with a mucosa (4.524 cm²) as a semi-permeable membrane. Rubber bands were used as a holding device.

To prepare the device according to the invention, 15 mL of the second medium, comprising a phosphate buffer with a pH of 7.4, was added to each well of a 6-deep-well cell culture plate using a dispensette to moisten the underground, and the culture plate was then preheated at 37 °C in an incubator. For filling the permeation cell according to the invention with 3 mL of diluted saliva substitute solution and the OTF containing ketamine HCl, one of the two openings was not completely closed with the mucosa. Furthermore, the permeation cell according to the invention was filled with a weight for weighting; individual lead balls with a weight of 5 g in total were used for this purpose. After closing the previously created opening with the mucosa and securing it with a rubber band, the permeation cell according to the invention was filled with 3 mL of diluted saliva substitute solution, the ketamine HCl-containing OTF, the weighting lead balls, and placed in the acceptor compartment with the openings closed with the mucosa as the donor compartment.

In order to determine the concentration, the preheated 6-deep-well plate was removed for the first replacement time of 3 minutes and the permeation cell according to the invention and the blank value were carefully placed in a well using a pair of tweezers. The same procedure was followed for the remaining replacement times of 6, 9, 12, 15, 18, 21, 24, 27 and 30 minutes. The loaded cell culture plate was then placed on a table shaker in the incubator and incubated at 37 °C and approx. 200 rpm. The collected samples were then analyzed using an HPLC system.

TABLE 4
Compilation of the measured values from Example 1, Example 2, Example
3 and comparison of the measured values from Example 1 with Example 3.

| | Flux at | | | | Normalized |
|---|---|---|---|---|---|
| | replacement | Average flux | Steady- | Permeability | permeated |
| | time 3 minutes | (Jₐ) | state flux | coefficient | amount |
| Example | [μg/cm² * min] | [μg/cm² * min] | (Jₛₛ) | (K_{P}) * | [μg/cm²] |
| 1 | 6.266 | 29.320 | 34.420 | 6.125 | 892 |
| 2 | 16.840 | 37.088 | 43.457 | 7.733 | 1136 |
| 3 | 15.87 | 40.014 | 51.170 | 9.105 | 1250 |
| Increase 1 | Factor 2.5 | 27% | 32% | 33% | 29% |
| vs 3 | | | | | |

1. A permeation cell, comprising a hollow body with at least two openings, characterized in that the at least two openings are each closed by at least one semi-permeable membrane.

2. The permeation cell according to claim 1, characterized in that the at least one semi-permeable membrane comprises a mucous membrane.

3. The permeation cell according to claim 1, characterized in that the hollow body has a volume of from 1 to 5 cm³.

4. The permeation cell according to claim 1, characterized in that the total area of the semi-permeable membranes is from 2 to 12 cm².

5. The permeation cell according to claim 1, characterized in that the hollow body comprises a material selected from the group of polymers.

6. The permeation cell according to claim 1, characterized in that the hollow body has the shape of a hollow cylinder, a hollow parallelepiped, a hollow cube, a hollow pyramid, a hollow prism, a hollow cone, and/or a hollow sphere, or a shape derived from these.

7. A device for measuring a permeation of molecules through at least one semi-permeable membrane, comprising a permeation cell according to claim 1 as a donor compartment, wherein the permeation cell contains a first medium comprising molecules, and an acceptor compartment containing a second medium, wherein the acceptor compartment is designed such that the second medium completely covers the semi-permeable membranes of the permeation cell.

8. The device according to claim 7, wherein the molecules comprise at least one pharmaceutically active ingredient.

9. The device according to claim 7, characterized in that the first medium comprising the molecules comprises a solution, dispersion, and/or suspension comprising at least one pharmaceutically active ingredient.

10. The device according to claim 7, characterized in that the second medium comprises at least a solvent and a buffer.

11. The device according to claim 7, characterized in that the donor compartment is designed such that it has a higher density in its entirety than the medium in the acceptor compartment.

12. A method for measuring a permeation of molecules through at least one semi-permeable membrane, comprising the steps of:
providing a permeation cell according to claim 1 as a donor compartment, wherein the permeation cell contains a first medium comprising molecules,
providing an acceptor compartment comprising a second medium,
placing the permeation cell in the acceptor compartment so that the semi-permeable membranes of the donor compartment are completely covered with the second medium.

13. The method according to claim 12, further comprising the step of taking a sample from the acceptor compartment to determine a concentration of the molecules that have diffused through the semi-permeable membranes of the donor compartment.

14. The method according to claim 13, characterized in that the sampling takes place over a period of 0.5 to 360 minutes.

15. (canceled)

16. The permeation cell according to claim 1, characterized in that the at least one semi-permeable membrane comprises a human oral mucous membrane.

17. The permeation cell according to claim 1, characterized in that the hollow body has a volume of from about 1.5 to about 3 cm³.

18. The permeation cell according to claim 1, characterized in that the total area of the semi-permeable membranes is from about 3 to about 9 cm³.

19. The permeation cell according to claim 1, characterized in that the hollow body has a volume of from 1 to 4 cm³ and a total area of the semi-permeable membranes from 2.5 to 10 cm².

20. The permeation cell according to claim 1, characterized in that the hollow body comprises polystyrene.

21. The method according to claim 14, wherein the samples are taken regularly.
